SEQUENCE LISTING (2 sequences)

SEQ ID NO: 1 - protein, 330 aa, Homo sapiens [sequence omitted]

SEQ ID NO: 2 - protein, 16 aa, Artificial
  Artificial linker sequence for conjugating two or more fusion peptides/proteins.
GGSGGSGGGGSGGGGS

(19) United States
(12) Patent Application Publication
(10) Pub. No.: US 2004/0198961 A1
(43) Pub. Date: Oct. 7, 2004

(54) Fce fusion proteins for treatment of allergy and asthma

(76) Inventors: Ling-Ling An, Boyds, MD (US); Herren Wu, Boyds, MD (US); Michael S.C. Fung, Houston, TX (US)

Correspondence Address: Cheryl Lijestrand, Tanox Inc, Suite 110, 10301 Stella Link, Houston, TX, 77025-5497, US

(21) Appl. No.: 10/479,326

(22) PCT Filed: Jun. 14, 2002
(86) PCT No.: PCT/US02/19448

Related U.S. Application Data
(62) Division of application No. 60/298,710, filed on Jun. 15, 2001.

(51) Int. Cl. C12Q 1/68; C07H 21/04; C07K 16/46; C12N 5/06

(52) U.S. Cl. 530/391.1; 435/69.7; 435/326; 435/320.1; 536/23.53

(57) ABSTRACT

The present invention includes Fcε fragments conjugated with FCγ fragments, for example, Fcε1-Hinge-FcE2-FcE3-FcE4-FCy; Hinge-FcE2-FcE3-FcE4-Fcy; FCε2-Fcε3-Fcε4-FCγ; FCε2-Fcε3-Fcγ; FCε3-Fcγ; and FCε3-Fcε4-FCγ, or any derivative or peptide, which has equivalent immunological function. The Fcγ fragment may be a fragment of any of the IgG subclasses (IgG1 I IgG2, IgG3, or IgG4), preferably IgG1 or IgG3, wherein the fragment binds FcγRIIB. The present invention also includes compositions suitable for administering to a patient suffering from an allergic disease comprising the fusion protein construct in a pharmaceutical composition including, for example, an excipient, diluant, or carrier. This treatment may be combined with anti-IgE therapy or allergen immunotherapy.

### CROSS REFERENCE TO RELATED APPLICATIONS

This application claims priority to U.S. Provisional Application 60/298,710 filed Jun. 15, 2001.

### FIELD OF THE INVENTION

This invention relates to fusion protein constructs comprising IgE Fcε fragments (natural and functional derivatives) and IgG Fcγ fragments. The constructs can bind to the high-affinity receptor (FcεRI) or the low-affinity receptor (FcεRII) and Fcγ receptors, such as FcγRIIB, resulting in the down-regulation of the expression of FcεRI on cell surfaces and blocking of IgE binding to these receptors.

### BACKGROUND OF THE INVENTION

The immune system provides a defense against infectious agents and tumors by reacting with antigens associated with bacteria, viruses, parasites, and tumor cells. However, not all immune responses are beneficial.

Allergic reactions result from immune responses to otherwise innocuous agents such as foods, drugs, and pollens, and can result in diseases such as asthma, allergic rhinitis, and food and drug allergies. These allergic diseases are usually associated with IgE-mediated hypersensitivity reactions, also known as immediate hypersensitivity, rather than delayed or cell-mediated hypersensitivity. These diseases affect as much as 10-40% of the population in industrialized countries. (Janeway, C A., et al., Immunology: the immune system in health and disease, Ed. 4, Elsevier Science Ltd., London; 1999). Asthma alone is estimated to affect more than 15 million Americans, with more than 5000 dying annually. More than 10% of children have allergic dermatitis at some point during their childhood (Leung, D., Mol. Gen. and Metab. 63:157-167 (1998). The prevalence of allergic diseases has risen dramatically over the last 20 years.

Allergic diseases are often, but not always, associated with high levels of circulating IgE. In IgE-mediated allergic reactions, IgE binds to FcεRI and if receptor-bound IgE interacts with allergens, it causes a cross-linking of IgE antibodies bound on effector cells, such as mast cells and basophils. As a result, the underlying receptors are aggregated, initiating an intracellular signal transduction cascade that triggers immediate degranulation and release of histamine and tryptase, followed by the synthesis and release of prostaglandins, leukotrienes, cytokines such as, IL-3, -4, -5, -6, -10 and -13, TNFα, and GM-CSF, as well as other mediators of allergic responses. These mediators cause the pathologic manifestations of allergic reactions.

There are two major receptors for IgE, the high affinity receptor FcεRI and the low-affinity receptor FcεRII. FcεRI forms either a trimeric αγ2 (one α-chain and two γ-chains) or a tetrameric αβγ2 (one α-chain, one β-chain, and two γ-chains) structure on the cell surface (Kinet, J P., Annu. Rev. Immunol. 1999, 17:931-972). The extracellular domains of the α-chain confer the ability of this receptor to bind IgE with high affinity (Kd=10⁻⁹−10⁻¹⁰M). Although the receptor is predominantly expressed on the surface of mast cells and basophils, low levels of FcεRI can also be found on human Langerhan's cells, dendritic cells, and monocytes, where it functions in IgE-mediated allergen presentation. In addition, FcεRI has been reported on human eosinophils and platelets (Hasegawa, S. et. al., Hematopolesis, 1999, 93:2543-2551). FcεRI is not found on the surface of B cells, T cells, or neutrophils. The expression of FcεRI on Langerhan's cells and dermal dendritic cells is functionally and biologically important for IgE-bound antigen presentation in allergic individuals (Klubal R. et al., J. Invest. Dermatol. 1997, 108 (3):336-42).

The low-affinity receptor, FcεRII (CD23) is a lectin-like molecule comprising three identical subunits with head structures extending from a long α-helical coiled stalk from the cellular plasma membrane (Dierks, A. E. et al., J. Immunol. 1993, 150:2372-2382). The IgE CH3 domains can bind two of the three head structures of FcεRII simultaneously with low affinity (Kd=6.3×10⁻⁷M). This IgE binding site, although located in the CH3 region, does not overlap with the FcεRI binding site. Upon binding to IgE, FcεRII associates with CD21 on B cells involved in the regulation of synthesis of IgE (Sanon, A. et al., J. Allergy Clin. Immunol. 1990, 86:333-344, Bonnefoy, J. et al., Eur. Resp. J. 1996, 9:63s-66s). FcεRII has long been recognized for allergen presentation (Sutton and Gould, 1993, Nature, 366:421-428). IgE bound to FcεRII on epithelial cells is responsible for specific and rapid allergen presentation (Yang, P. P., J. Clin. Invest, 2000, 106:879-886). FcεRII is present on several cell types, including B-cells, eosinophils, platelets, natural killer cells, T-cells, follicular dendritic cells, and Langerhan's cells.

The structural entities on the IgE molecule that interact with FcεRI and FcεRII have also been identified. Mutagenesis studies have indicated that the CH3 domain mediates IgE interaction with both FcεRI (Presta et al., J. Biol. Chem. 1994, 269:26368-26373; Henry A. J. et al., Biochemistry, 1997, 36:15568-15578) and FcεRII (Sutton and Gould, Nature, 1993, 366: 421-428; Shi, J. et al., Biochemistry, 1997, 36:2112-2122). The binding sites for both high- and low-affinity receptors are located symmetrically along a central rotational axis through the two CH3 domains. The FcεRI-binding site is located in a CH3 domain on the outward side near the junction of the CH2 domain, whereas the FcεRII-binding site is on the carboxyl-terminus of CH3.

Early studies of human basophil density showed a correlation between the level of IgE in the plasma of a patient and the number of FcεRI receptors per basophil (Malveaux et al., J. Clin. Invest., 1978, 62:176). They noted that the FcεRI densities in allergic and non-allergic persons range from 10⁴ to 10⁶ receptors per basophil. Later it was shown that treatment of allergic diseases with anti-IgE decreased the amount of circulating IgE to 1% of pretreatment levels (MacGlashan et al., J. Immunol., 1997, 158:1438-1445). MacGlashan analyzed serum obtained from patients treated with whole anti-IgE antibody, which binds free IgE circulating in the serum of the patient. They reported that lowering the level of circulating IgE in a patient resulted in a lower number of receptors present on the surface of basophils. Thus, they hypothesized that FcεRI density on the surface of basophils and mast cells is directly or indirectly regulated by the level of circulating IgE antibody.

More recently, WO 99/62550 disclosed the use of IgE molecules and fragments, which bind to FcεRI and FcεRII IgE binding sites to block IgE binding to receptors. However, effective therapies that lack deleterious side effects for the management of these allergic diseases are limited. One therapeutic approach to treating allergic diseases involved using humanized anti-IgE antibody to treat allergic rhinitis and asthma (Come, J. et al., J. Clin. Invest. 1997, 99:879-887; Racine-Poon, A. et al., Clin. Pharmcol. Ther. 1997, 62:675-690; Fahy, J. V. et al., Am. J. Resp. Crit. Care Med. 1997, 155:1824-1834; Boulet, L. P. et al., Am. J. Resp. Crit. Care Med., 1997, 155:1835-1840; Milgrom, E. et al., N. Engl. J. Med., 1999, 341:1966-1973). These clinical data demonstrate that inhibition of IgE binding to its receptors is an effective approach to treating allergic diseases.

Advantageously, an Fcε-Fcγfusion protein can cross-link FcεRI (via Fcε) and FcγRIIB (via Fcγ) on mast cells and basophils. The result of this receptor cross-linking will inhibit degranulation of these cells. This inhibition is thought to involve the tyrosine phosphorylation of immunoreceptor tyrosine-based inhibitory motifs (ITIMs) in the FcγRIIB cytoplasmic tail, creating binding sites for SH2-containing protein (Src homology domain containing protein tyrosine phosphatase 1 and 2 (SHP-1, SHP-2)) and/or lipid (SH2 domain-containing polyphosphatidyl-inositol 5-phosphatase) phosphatases that oppose activating signals from the antigen receptor FcεRI, which bears an immunoreceptor tyrosine-based activating motif (ITAM) in the cytoplasmic tail. The Fcε-Fcγfusion proteins can also bind to FcεRII on B cells, Langerhans cells, dendritic cells and monocytes, thereby blocking the presentation of allergens (complexed with IgE) to these cells via FcεRI and/or FcεRII. This will reduce immune responses to allergens which include IgE production from B cells and generation of allergen-specific T cells.

This invention provides a treatment for IgE-mediated allergic diseases by down-regulating IgE production, reducing the degranulation of mast cells and basophils, and decreasing the expression of FcεRI receptors on mast cells and basophils. The invention also provides a means to reduce immune response to allergens by preventing allergen presentation to Langerhans cells and dendritic cells via FcεRI and to B cells via FcεRII.

### SUMMARY OF THE INVENTION

The present invention includes fusion proteins comprising IgE FCε fragments and IgG Fcγ fragments, for example, Fcε1-Hinge-Fcε2-Fcε3-Fcε4-Fcγ; Hinge-Fcε2-Fcε3-Fcε4-Fcγ; Fcε2-Fcε3-Fcε4-Fcγ; Fcε2-Fcε3-Fcγ; Fcε3-Fcγ; and Fcε3-Fcε4-Fcγ, or any modified fragment or peptide that has an equivalent immunological function capable of binding to the FcεRI or FcεRII and FcγRIIB receptors. The Fcγ fragment may be a fragment from any of the IgG subclasses (IgG1, IgG2, IgG3, or IgG4) and may comprise Fcγ1-Fcγ2-Fcγ3 or any smaller fragment thereof capable of binding the FcγRIIB receptor. The Fcγfragment can be linked to the Fcεfragment via its C- or N-terminus. The Fcεfragment and Fcγfragment may be conjugated directly to each other or through a linker.

The fusion protein binds to FcεRI on basophils and mast cells and FcεRII on B-cells, which results in blocking of IgE binding to these receptors, and down regulating the expression of these receptors on the surface of basophils, mast cells, and B-cells. The fusion proteins can cross-link FcεRI and FcγRIIB on mast cells and basophils and thereby reduce the degranulation of these cells. Since there is no allergen-binding region present on the Fcε fragments of the fusion proteins, there can be no cross-linking of the fragments by allergen molecules on the surface of mast cells or basophils, hence no aggregation of receptors and no initiation of the intracellular signal transduction cascade that triggers the release of the mediators responsible for the allergic response. Moreover, the binding of Fcεαfragments to the FcεRI receptor prevents binding of IgE, thereby eliminating allergen specific immune responses.

The present invention also includes compositions suitable for administering to a patient suffering from an allergic disease comprising the fusion protein construct in a composition including, for example, an excipient, diluent, or carrier. This treatment may also be combined with anti-IgE therapy or allergen immunotherapy.

The present invention includes nucleic acid sequences and vectors encoding the fusion proteins, as well as host cells transfected with these sequences. These mammalian sequences include mouse, rat, rabbit, canine, feline, ovine, porcine, equine, and human.

The present invention includes a method of ameliorating or preventing an IgE-mediated allergic response in a susceptible mammalian subject, comprising administering an effective amount of the fusion protein comprising Fcε fragments and Fcγfragments to the mammalian subject. The allergic response may be associated with allergic asthma, allergic rhinitis, hay fever, food allergies, such as peanut or tree nut allergies, atopic dermatitis or drug allergy.

### BRIEF DESCRIPTION OF THE FIGURES

FIG. 1 is a schematic diagram of the epsilon heavy chain of human IgE. The amino acid positions correspond to the numbering system given by Bennich (see below).

FIG. 2 shows the reactivity of Fcε-Fcγ with recombinant human soluble FcεRIa in ELISA.

FIG. 3 shows the inhibition of biotinylated IgE binding to FcεRIα by Fcε-Fcγ in ELISA.

FIG. 4 shows the inhibition of Fcε-Fcγ binding to FcεRIα by IgE in ELISA.

FIG. 5 shows the amino acid sequence of Fcγ.

### DETAILED DESCRIPTION OF THE INVENTION

The present invention includes fusion proteins wherein an IgE Fcε fragment, such as Fcε1-Hinge-Fcε2-Fcε3-Fcε4; Hinge-Fcε2-Fcε3-Fcε4; Fcε2-Fcε3-Fcε4; Fcε2-Fcε3; Fcε3; and Fcε3-Fcε4, is conjugated with an IgG Fcγ fragment, such as Fcγ2-Fcγ3 or Fcγ2, and may be of any subtype IgG1, IgG2, IgG3, or IgG4, preferably IgG1 or IgG3. These fusion proteins can bind to FcεRI and/or FcεRII receptors, thus blocking IgE binding to FcεRI and FcεRII, as well as to Fcγ receptors, specifically the FcγRIIB receptor. Because the fusion protein includes only immunoglobulin Fc regions and no antigen-binding portions of the immunoglobulin, contact with an allergen cannot result in cross-linking and degranulation of mast cells or basophils.

The Fcεfragments may be conjugated to the Fcγ fragment via various linkers well known in the art. An example of a suitable linker is a nonimmunogenic 16 amino acid linker having the sequence (GGS)₂ joined with (GGGGS)₂ (Argos, P., 1990, J. Mol. Biol. 211:943; Huston, J. S. et al. 1988, Proc. Natl. Acad. Sci. USA 85:5879). The linker should be designed to be nonimmunogenic to the mammalian subject receiving the fusion protein.

It has been shown that when treating patients with anti-IgE monoclonal antibodies and the amount of circulating IgE was significantly reduced, the number of FcεRI receptors was concomitantly reduced (Chang, “The pharmacological basis of anti-IgE therapy”, Nat Biotechnol. 2000 February;18(2):157-62.) The binding of fusion proteins of the present invention to the FcεRI receptor down-regulates FcεRI expression, thereby reducing the severity or preventing future allergic responses, as fewer receptors would be present. Such down-regulation may also reduce allergen presentation by professional antigen-presenting cells such as dendritic cells.

If the Fcγfragment's subclass is IgG1 or IgG3, the fusion proteins of the present invention can crosslink FcεRI brearing a cytoplasmic ITAM motif, and FcγRIIB bearing a cytoplasmic ITIM motif on mast cells and basophils, thereby preventing degranulation of these cells. Alternatively, the Fcγmay be of the IgG2 or IgG4 subclass. Regardless of the Fc subclass to be used, the fusion constructs will have a much longer biological half-life in the host than Fcε fragments alone. This is advantageous for therapeutic intervention of chronic diseases like allergy and asthma.

In addition, the fusion protein may comprise Fcε2-Fcε3-Fcγ3 and bind to FcεRI and FcεRII receptors. The Fcεfragment may also comprise the hinge region of IgE-Fcε2-Fcε3-Fcε4 or a functional fragment thereof. The fusion proteins of the invention bind to the FcεRI and/or FcεRII receptors with at least 75% binding affinity of native IgE, 85% binding affinity, 95% binding affinity, or with greater binding affinity than native IgE.

The Fcεand Fcγfragments include natural and synthetic fragments, as well as proteins with different sequences but equivalent immunological function to the fusion protein. Such immunologically equivalent proteins would have the ability to bind both FcεRI and FcεRII, as well as FcγRIIB. Immunologically equivalent proteins can be made by any of a number of well-known techniques, including starting with a protein combinational library and isolating binding proteins, followed by optimizing their binding affinity for FcεRI, FcεRII, and FcγRIIB or by making selective alterations to the fusion protein which include the Fcε and Fcγ fragments. Methods for altering the amino acid sequence of antibody fragments are well known in the field. One method commonly used for introducing random mutations into antibody genes in vitro involves the use of error-prone polymerase and affinity selection (Hawkins, R. et al., J. Mol. Biol., 1992, 226:889-896). Functionally equivalent fusion proteins may include, but are not limited to, additions, substitutions, deletions, and modifications of amino acid residues within the amino acid sequence encoded by the fusion protein nucleotide sequences, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

While random mutations can be made to fusion protein encoding DNA (using random mutagenesis techniques well known to those skilled in the art) and the resulting mutant tested for activity, site-directed mutations of the coding sequence can be engineered (using site-directed mutagenesis techniques well known to those skilled in the art) to generate mutant fusion proteins with increased function, e.g., higher receptor binding affinity, decreased function, and/or increased physiological half-life. One starting point for such analysis is by aligning the FCE fragment or Fcγfragment sequences with corresponding gene/protein sequences from, for example, other mammals in order to identify amino acid sequence motifs that are conserved between different species. Non-conservative changes can be engineered at variable positions to alter function, binding affinity, or both. Alternatively, where alteration of function is desired, deletion or non-conservative alterations of the conserved regions (i.e., identical amino acids) can be engineered. For example, deletion or non-conservative alterations (substitutions or insertions) of the various conserved domains, keeping in mind the potential for immunogenicity.

Other modifications to the coding sequence can be made to generate fusion proteins that are better suited for expression, scale up, etc. in the host cells chosen. For example, cysteine residues can be deleted or substituted with another amino acid in order to eliminate disulfide bridges; N-linked glycosylation sites can be altered or eliminated to achieve, for example, expression of a homogeneous product that is more easily recovered and purified from yeast hosts which are known to hyperglycosylate N-linked sites.

The present invention provides compositions that comprise the fusion proteins of the invention and physiologically acceptable excipients, additives, carriers or diluents. Suitable physiological agents are described in Remington's Pharmaceutical Sciences, A. Osol, a reference commonly used in this field. In carrying out methods of the present invention, conjugated compounds of the present invention can be used alone or in combination with other therapeutic or additional agents. Such additional agents include excipients such as coloring, stabilizing agents, osmotic agents and antibacterial agents.

For parenteral administration, the fusion proteins of the invention can be, for example, formulated as a solution, suspension, emulsion or lyophilized powder in association with a physiologically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of active ingredient in 0.9% sodium chloride solution. The compositions according to the present invention may be administered as a single dose or in multiple doses. The compositions of the present invention may be administered either as individual therapeutic agents or in combination with other therapeutic agents. The treatments of the present invention may be combined with conventional therapies, which may be administered separately, sequentially or simultaneously.

The compositions of the present invention may be administered by any means that enables the active agent to reach the targeted cells. These methods include, but are not limited to, oral, topical, intradermal, subcutaneous, intravenous, intramuscular and intraparenteral modes of administration. The compounds may be administered singularly or in combination with other compounds. The physiologically acceptable agent to be added is normally selected on the basis of the route of administration and standard pharmaceutical practice.

Fusion proteins may be prepared, e.g., by recombinant DNA techniques or by chemical conjugation by forming a covalent bond or other techniques well known in the art for generating fusion proteins. Provision of a suitable DNA sequence encoding the desired fusion protein permits the production of the fusion protein using recombinant techniques well known in the art. The coding sequence can be obtained from natural sources or synthesized or otherwise constructed using widely available starting materials by routine methods. When the coding DNA is prepared synthetically, advantage can be taken of known codon preferences of the intended host where the DNA is to be expressed.

To produce fusion proteins of the invention, one having ordinary skill in the art can, using well known techniques, synthesizes or obtains a DNA molecule encoding the Fcε fragment or portions thereof conjugated with a DNA molecule encoding Fcγ fragment or portions thereof from readily available human DNA and insert that DNA molecule into a commercially available expression vector for use in well known expression systems. Included in these systems are those in which the fusion protein of interest is produced as a single chain.

There are numerous examples of commercially available plasmids from companies such as Invitrogen, San Diego, Calif., which may be used for recombinant production in E. coli, production in S. cerevisiae strains of yeast, or in a complete baculovirus expression system used for production in insect cells. There are also commercially available vectors for production in mammalian cells such as Chinese Hamster Ovary cells and NS/O cells.

One having ordinary skill in the art may use these commercially available expression vectors and systems or produce vectors using well known methods and readily available starting materials. Expression systems containing the requisite control sequences, such as promoters and polyadenylation signals, and preferably enhancers, are readily available and known in the art for a variety of hosts. See e.g., Sambrook et al., Molecular Cloning a Laboratory Manual, Second Ed. Cold Spring Harbor Press (1989). Thus, the desired proteins can be prepared in either prokaryotic and eukaryotic systems, resulting in a spectrum of processed forms of the protein.

A wide variety of eukaryotic hosts are available for production of recombinant foreign proteins. As in bacteria, eukaryotic hosts may be transformed with expression systems that produce the desired protein directly, but more commonly signal sequences are provided to effect the secretion of the protein. Eukaryotic systems have the additional advantage that they are able to process introns which may occur in the genomic sequences encoding proteins of higher organisms. Eukaryotic systems also provide a variety of processing mechanisms which result in, for example, glycosylation, carboxy-terminal amidation, oxidation or derivatization of certain amino acid residues, conformational control, and so forth.

Commonly used eukaryotic systems include, but is not limited to, yeast, fungal cells, insect cells, mammalian cells, avian cells, and cells of higher plants. Suitable promoters are available which are compatible and operable for use in each of these host types as well as are termination sequences and enhancers, as e.g. the baculovirus polyhedron promoter. As above, promoters can be either constitutive or inducible.

In some embodiments, a DNA molecule that includes a nucleotide sequence that encodes the fusion protein of the invention is synthesized using the amino acid sequence information herein and the genetic code. Those having ordinary skill in the art can readily synthesize DNA molecules that include nucleotide sequences that encode the fusion proteins of the invention using codons preferred by a desired host cell. The DNA molecule that is generated may be inserted in an expression vector which allows for very high levels of expression, sometimes referred to as overexpression, in a desired host.

The particulars for the construction of expression systems suitable for desired hosts are known to those in the art. For recombinant production of the protein, the DNA encoding it is suitably ligated into the expression vector of choice and then used to transform the compatible host which is then cultured and maintained under conditions wherein expression of the foreign gene takes place. The protein of the present invention thus produced is recovered from the culture, either by lysing the cells or from the culture medium as appropriate and known to those in the art.

One having ordinary skill in the art can, using well known techniques, isolate the protein that is produced.

It will be apparent to a skilled person in the field that a therapeutic composition can be provided in the form of an oral liquid, tablet, or capsule, nasal spray, aerosol, suspension, solution, emulsion, and/or eye drop. Alternative delivery methods include injection, nebulization, or inhalation. The appropriate dosage can be extrapolated from the dosages that indicate efficacy in vitro or in animal studies. The dosage administered varies depending upon factors such as: pharmacodynamic characteristics; its mode and route of administration; age, health, and weight of the recipient; nature and extent of symptoms; kind of concurrent treatment; and frequency of treatment. Usually, the dosage of fusion protein can be about 1 to 3000 milligrams per 50 kilograms of body weight; 10 to 1000 milligrams per 50 kilograms of body weight; or 25 to 800 milligrams per 50 kilograms of body weight. Ordinarily 8 to 800 milligrams are administered to an individual per day in divided doses 1 or more times per day is effective to obtain desired results.

### EXAMPLES OF Fc FRAGMENTS

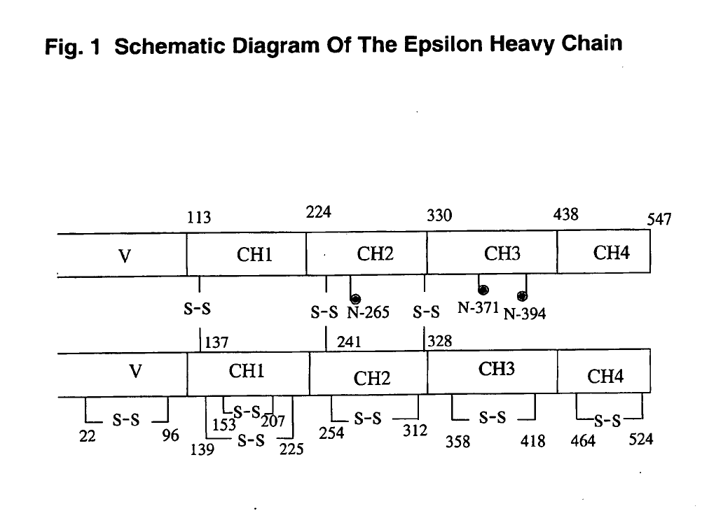

The schematic diagram of the epsilon chain of human IgE is shown in FIG. 1, which corresponds to the numbering system given by Bennich (Progress in Immunology II, 1974, 1:49-58). There are five domains, a variable domain, and four constant domains CH1-CH4, having a sequence of approximately 550 amino acids.

(1) Fcε1-Fcε2-Fcε3-Fcε4-Fcγ

In this example, the fragment encompasses a peptidic segment starting from amino acid 113 to amino acid 547 of IgE. Cys-225 forms a disulfide bond with Cys-139 in the CH1 region (FIG. 1).

(2) Fcε2-Fcε3-Fcε4-Fcγ

The fragment encompasses a peptidic segment from amino acid 224 to amino acid 547 of IgE. Cys-225 may be converted to another amino acid, e.g., Ala-225 (Young R. J. et al., Protein Engineering, 1995, 8:193-199) to avoid the formation of disulfide bonds with other free sulfhydryl groups within the Fcε region.

(3) Fcε2-Fcε3-Fcγ

The fragment encompasses a peptidic segment from amino acid 224 to amino acid 437 of IgE. As described above, Cys-225 may be converted to, e.g., Ala-225.

(4) Fcε3-Fcγ

The fragment encompasses a peptidic segment from amino acid 328 to amino acid 437 of IgE. The fragment includes two amino acids from Fcε2, Cys-328, and Val-329. Cys-328 provides a disulfide bond to form a dimer.

(5) Fcε3-Fcε4-Fcγ

The fragment encompasses a peptidic segment from about amino acid 328 to amino acid 547 of IgE.

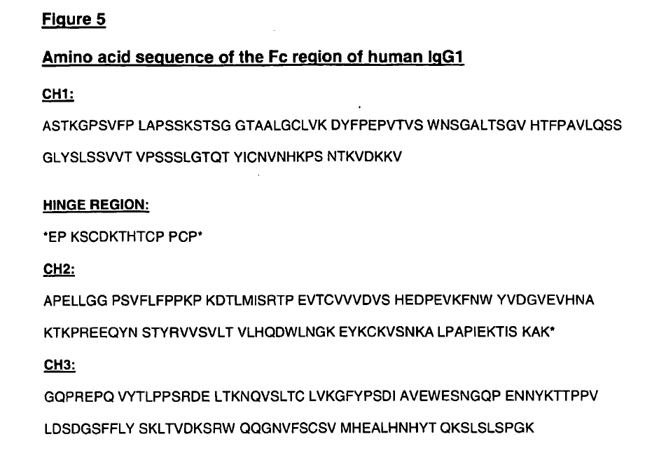

The deduced amino acid sequence from the nucleotide sequence of a human immunoglobulin constant region of IgG1 gene (from Nucleic Acid Research 10:4071 (1982) Ellison, J W; Berson, B J and Hood L E) is shown in FIG. 5. The Fcγportion of the fusion protein can comprise the entire sequence or any fragment that binds to the FcγRIIB receptor, such as the Fcγ2-Fcγ3 fragment or the Fcγ2 fragment.

Preparation OF Fusion Constructs

The Fcε cDNA sequence was obtained from total RNA isolated from human IgE expressing cell line, SE44 (Sun, L K. et al., J Immunol. 1991, 146:199-205) by using RT-PCR. The Fcε fragments were conjugated to Fcγfragments (different subclasses) with the 16 amino acid linker described above and cloned into pcDNA3.1. The plasmids were transfected into 293T cell line for transient expression of the fusion proteins. The fusion proteins were purified by protein A chromatography and then tested for activities in different assays (see below). Functional constructs were expressed in a stable mammalian cell line, for example, CHO.

Characterization of Fusion Constructs

### Example 1

### Reactivity of Fcε-Fcγ4 with FcεRIα in ELISA

The binding of four purified Fcε-Fcγ4 fusion proteins bearing the Fcεfragments (Fcε2-Fcε3-Fcε4, Fcε2-Fcε3, Fcε3, or Fcε3-Fcε4) to recombinant human FcεRIα was tested by ELISA. Each well of Immunlon I microtest plates (Dynatech Laboratories, Chantilly, Va.) was coated overnight with 50 μl of purified baculovirus-expressed human soluble FcεRIα (Heska, Fort Collins, Colo.) at 0.2 μg/ml in sodium carbonate buffer pH 9.6. The non-specific binding sites in the wells were then saturated by incubation with 200 μl of 2% BSA in PBS for one hour. The wells were then washed with PBST buffer (PBS containing 0.05% TWEEN® 20). Fifty microliters of each fusion protein (1 nM) in BSA were added to each well for one hour at room temperature. The wells were washed with PBST. The bound fusion proteins were then detected by reaction with diluted horseradish peroxidase (HRP) conjugated mouse anti-human IgG4 (Fc specific) (Sigma, St. Louis, Mo.) for one hour at room temperature. The wells were then washed with PBST. Peroxidase substrate solution containing 0.1% 3,3,5,5, tetramethyl benzidine (Sigma) and 0.003% hydrogen peroxide (Sigma) in 0.1M sodium acetate pH 6.0 was added to the wells for color development for 30 minutes. The reaction was terminated by addition of 50 μl of 0.5 M H₂SO₄ per well. The optical density (OD) was read at 450 nm with an ELISA reader (Dynatech).

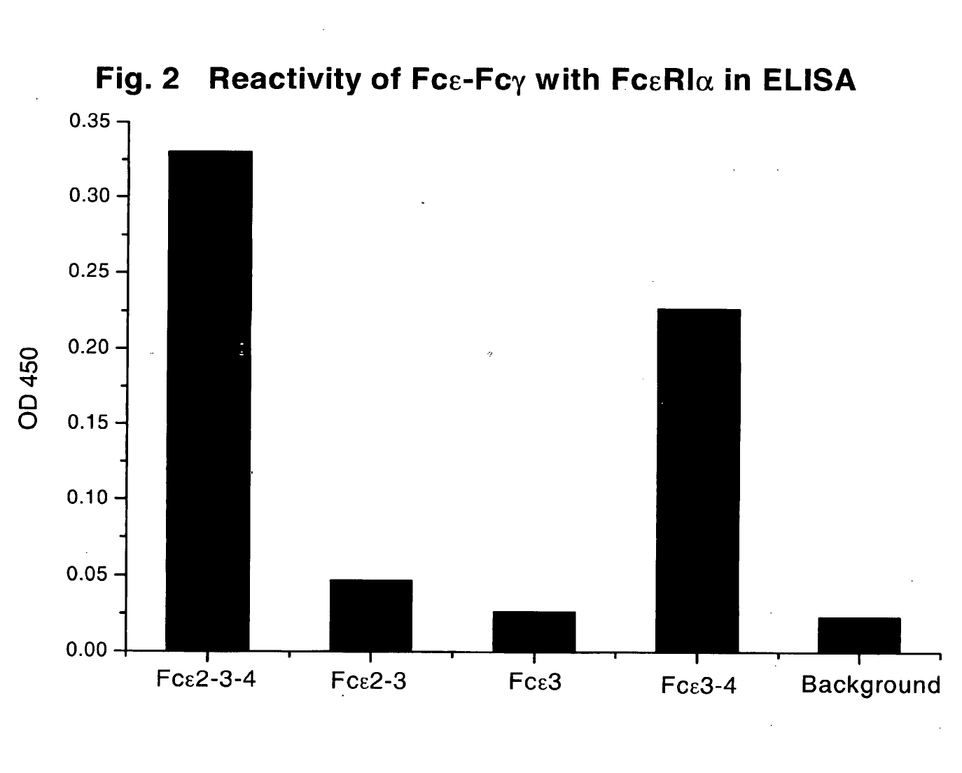

The results show that Fcε2-Fcε3-Fcε4 and Fcε3-Fcε4 bind strongly to recombinant human soluble FcεRIα, whereas Fcε2-Fcε3 and Fcε3 bind weakly to the protein (FIG. 2), indicating that both Fcε2-Fcε3-Fcε4 and Fcε3-Fcε4 contain the epitopes essential for the binding of IgE to FcεRIα.

### Example 2

### Inhibition of Biotinylated IgE Binding to FcεRIα by Fcε-Fcγ In ELISA

The competition of the binding of biotinylated IgE to recombinant human soluble FcεRIα by the four Fcε-Fcγ fusion proteins was tested by ELISA. Each well of Immunlon I microtest plates (Dynatech) was coated overnight with 50 μl of purified baculovirus-expressed human soluble FcεRIα (Heska, Fort Collins, Colo.) at 0.2 μg/ml in sodium carbonate buffer pH 7.4. The non-specific binding sites in the wells were then saturated by incubation with 200 μl of 2% BSA in PBS for one hour. The wells were then washed with PBST buffer (PBS containing 0.05% TWEEN® 20). Increasing amounts of the fusion proteins were mixed with a constant amount of pre-titered biotinylated recombinant human IgE (1 nM) at molar ratios of 0.065, 0.125, 0.25, 0.5, 1, 2, and 4 (Fcε-Fcγ:IgE). Unlabeled IgE was used as positive control. Fifty microliters of the mixture were added to each well for incubation at room temperature for 1 hour. The wells were then washed with PBST buffer. Bound biotinylated IgE was then detected by adding 50 ml of streptavidin conjugated HRP (Jackson ImmunoResearch Lab, West Grove, Pa.) for incubation at room temperature for one hour. The wells were then washed. Peroxidase substrate was then added for color development as described above. The OD was measured at 450 nm by an ELISA plate reader.

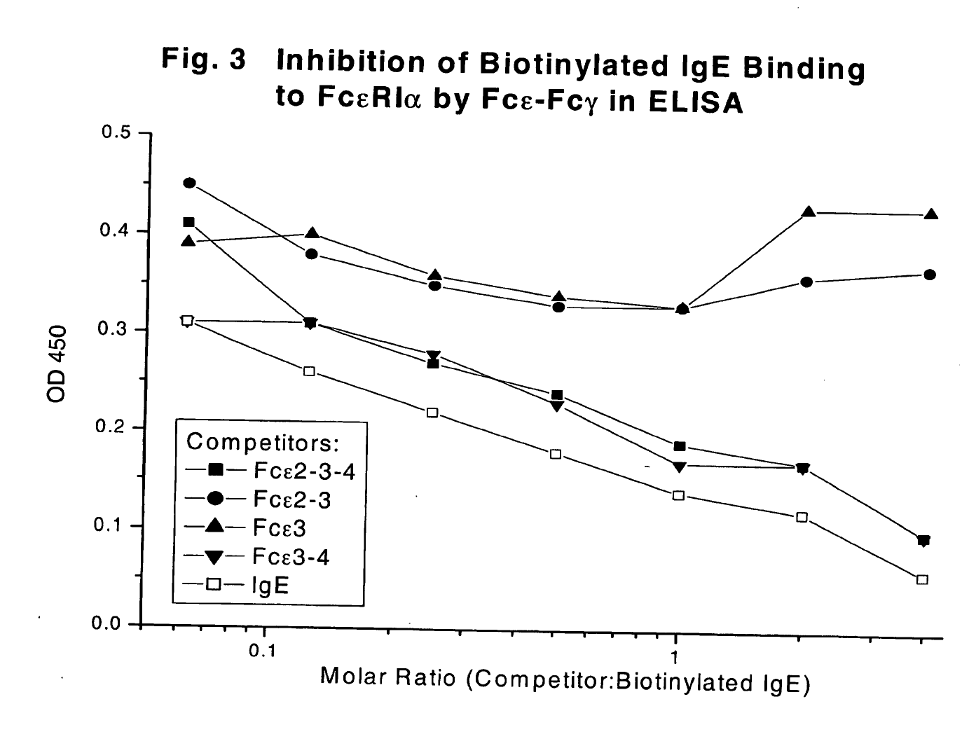

The results show that both Fcε2-Fcε3-Fcε4 and Fcε3-Fcε4 compete with the binding of biotinylated IgE to FcεRIα as effectively as unlabeled IgE, as indicated by the similar slope of the dose-dependent competition curves (FIG. 3). Fcε2-Fcε3 and Fcε3 do not show any competition as expected, because these two proteins bind weakly to FcεRIα as shown in FIG. 2.

### Example 3

### Inhibition of Fcε-FcγBinding to FcεRIα by IgE in ELISA

To confirm the results in Example 2, the inhibition of the binding of Fcε2-Fcε3-Fcε4 and Fcε3-Fcε4 to FcεRIα by IgE was also tested by ELISA. Each well of Immunlon I microtest plates (Dynatech) was coated overnight with 50 μl of purified baculovirus-expressed human soluble FcεRIα (Heska, Fort Collins, Colo.) at 0.2 μg/ml in sodium carbonate buffer pH 7.4. The non-specific binding sites in the wells were then saturated by incubation with 200 μl of 2% BSA in PBS for one hour. The wells were then washed with PBST buffer (PBS containing 0.05% TWEEN® 20). Increasing amounts of recombinant IgE were mixed with a constant amount of Fcε2-Fcε3-Fcε4 or Fcε3-Fcε4 (1 nM) at molar ratios of 0.125, 0.25, 0.5, 1, 2, 4, 8, and 16 (IgE:Fcε-Fcγ). Fifty microliters of the mixture were added to each well for incubation at room temperature for 1 hour. The wells were then washed with PBST buffer. The bound fusion proteins were then detected by reaction with diluted horseradish peroxidase (HRP) conjugated mouse anti-human IgG4 (Fc specific) (Sigma) for one hour at room temperature. The wells were then washed with PBST. Peroxidase substrate was then added for color development as described above. The OD was measured at 450 nm by an ELISA plate reader.

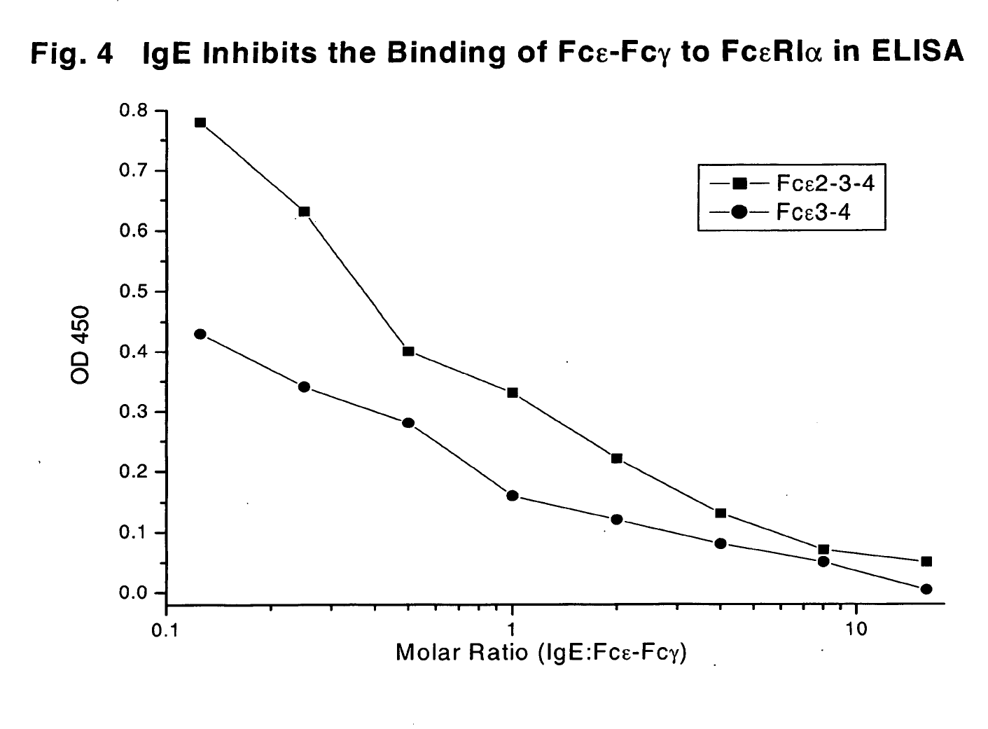

The results show that IgE competes effectively the binding of Fcε2-Fcε3-Fcε4 and Fcε3-Fcε4 to FcεRIα (FIG. 4). Together, the data from FIG. 3 and FIG. 4 indicate that Fcε2-Fcε3-Fcε4 and Fcε3-Fcε4 retain the FcεRIα-binding property of native IgE.

### Example 4

### Functional Assays with Human Basophils

A. Preparation of Human Basophils

Approximately 40 ml of venous blood collected from healthy individuals was diluted in three volumes of ice-cold phosphate-buffered saline (PBS) and 35 ml aliquots of this solution were carefully layered over 15 ml of Ficoll Paque. After density gradient centrifugation, the upper layer was aspirated leaving the mononuclear cell layer undisturbed at the interphase. The mononuclear cell layers were pooled and washed with EDTA-PBS. Cell numbers were determined by trypan blue staining and the final volume adjusted to 10⁶ per μl.

Basophils were then further purified using a midi-MACS column (Miltenyi Biotec, Auburn, Calif.). The kit provided a Hapten-Antibody cocktail containing hapten-conjugated anti-CD3, CD7, CD14, CD15, CD16, CD36, CD45A, and anti-HLA-DR antibodies. The colloidal super-magnetic MACS microbeads were conjugated with monoclonal anti-hapten antibody. Non-basophilic cells were indirectly magnetically labeled by using the cocktail and the magnetically labeled cells were removed by retaining them on the MACS column in a magnetic field.

The purified basophils were then evaluated by flow cytometry. Samples to be evaluated were blocked with human IgG (1 μg/ml 10⁵ cells). The following individual labels were used: goat anti-human IgE-FITC, mouse anti-human CD123-PE, mouse IgG1-PE, mouse anti-human CD45-PE, and mouse anti-HLA-DR-FITC. The following double labels were used: (1) goat anti-human IgE-FITC and goat anti-CD45-PE and (2) mouse anti-CD123-PE and mouse anti-HLA-DR-FITC. After the respective samples were stained, FACS analysis was performed.

B. IgE-Mediated Degranulation Of Basophils

To assure that each fusion protein did not result in the release of mediators, a histamine release assay was performed using standard reagents and protocols obtained, for example, from Beckman Coulter (Fullerton, Calif.). Briefly, cells were sensitized with IgE in the presence or absence of the fusion protein to be tested. In this example, a recombinant IgE (rIgE), which consists of variable regions specific to a peptidic segment derived from HIV-1 gp120 V3 region and the constant region of IgE, was used. The sensitized cells were then challenged with (1) the positive control, an ovalbumin conjugate of HIV-1 gp120 V3 peptides, (2) an anti-IgE, C5a, F-met mixture, or (3) IgG. The amount of histamine released was measured and used to measure basophil degranulation by the fusion proteins.

C. FcεRI Expression on Basophils

FcεRI expression of basophils will be studied by FACS analysis. Basophils are first cultured for 14 days in Iscove's modified Dulbeco's media containing 2% heat inactivated fetal calf serum, 40 μg/ml gentamicin, and 10 ng/ml rIL-3. Cell viability is tested at day 7 and day 14 using trypan blue stain.

After culturing for 14 days, an aliquot of 0.5×1 06 basophils is mixed with one of the following reagents in varying concentrations (10-1000 ng/ml) and incubated for 7 days: (1) rIgE alone, (2) rIgE+Fcε1-Fcε2-Fcε3-Fcε4-Fcγ; (3) rIgE+Fcε2-Fcε3-Fcε4-Fcγ; (4) rIgE+Fcε3-Fcε4-Fcγ; (5) rIgE+Fcγ3-Fcγ; (6) rIgE+Fcε2-Fcε3-Fcγ; (7) rIgE+IgG-γ4; (8) cells without rIgE. Also as a control, cells were cultured in the presence of each individual Fcε-Fcγ fusion protein, but in the absence of rIgE.

Cells were then centrifuged, washed, and blocked with IgG to reduce non-specific binding of antibodies to FcγR. Each sample was then stained with anti-FcεRI mAb-FITC or a mouse iso-type control and subjected to FACS analysis. Those cells that were not exposed to IgE (#8 above) served as a baseline for the number of receptors present on the cultured basophils. Cells exposed to rIgE in the absence of fusion protein provided a positive control for maximal FcεRI expression (#1 above).

An increase in the number of FcεRI receptors over the negative control was measured by an increase in the level of FITC staining using the anti-FcεR1 mAb, which binds to an epitope distinct from that of IgE. Those fragments exhibiting low levels of FITC staining are suitable candidates for treating an allergic reaction.

Exhibit 5: Inhibitory Effect of Fcε-FcγFusion Proteins on IgE-Mediated Histamine Release From Cultured Human Mast Cells

A. Mast Cell Culture:

Cryopreserved human cord blood CD34⁺ cells (BioWhittaker Inc., Walkersville, Md.) were cultured for 8 weeks in a culture medium consisting of RPMI 1640 (GIBCO-BRL, Rockville, Md.) supplemented with 10% fetal bovine albumin (Sigma-Aldrich, St. Louis, Mo.), 2 mM L-glutamine, 50 mM 2-mercaptoethanol, 100U/ml penicillin, 100 mg/ml streptomycin, 10 mg/ml gentamycin (GIBCO/BRL), 100 ng/ml stem cell factor, 50 ng/ml IL-6 and 5 ng/ml IL-10 (R&D Systems Inc., Minneapolis, Minn.). The cytokine-supplemented medium was replaced once a week and the adherent fraction of cells was discarded by transferring the non-adherent cells to new culture flasks. During the third week CD14 & CD15 positive cells were depleted using beads coated with CD14 & CD15 specific monoclonal antibodies (M-450 CD14, M-450 CD15) according to manufacturers protocol (Dynal, Lake Success, N.Y.). Aliquots of cultured cells were taken and monitored for tryptase expression weekly for 8 weeks.

B. Immunocytochemistry:

Every week aliquots of 3-4×10⁴ cultured cells were spun onto glass slides in a cytocentrifuge. Cells on the slide were air dried, and fixed in Carnoy's fluid (60% ethanol, 30% chloroform, and 10% glacial acetic acid) for 10 min at room temperature. After washing with PBS, slides were blocked with blocking buffer (3% BSA, 1.5% normal horse serum, 0.2% Triton X-100, 0.02% NaN₃, in PBS) for 30 min at room temperature. After blocking, the slides were incubated with mouse anti-human tryptase MAb (alkaline phosphatase conjugated, Chemicon, Temecula, Calif.) for 1 hr at 37° C. The antibody was diluted 1:300 in a PBS buffer containing 3% BSA, 0.2% Triton X-100, 0.02% NaN₃. Slides were washed in PBS (containing 0.2% Triton X-100) and immunocytochemical procedures were carried out using the alkaline phosphatase substrate kit (Vector Lab Inc., Burlingame, Calif.). Cells exhibiting strong immunoreactivity were counted and expressed as a percentage of total cells counted.

C. Flow Cytometry:

On week 7 cells were washed once with PBS then pre-incubated with cold PBS supplemented with 1% BSA and 1% human gamma globulin (PBSBH) for 30 min, 4° C. Then cells were incubated with PE/FITC-conjugated mouse anti-human MAbs specific for the following epitopes (MAbs were purchased from BD PharMingen): c-kit (the receptor for stem cell factor), CD13 (a marker for aminopeptidase N), CD14 (marker for monocytes), CD16 (neutrophil marker) and CD61 (P3 subunit of the integrin family). Cells were incubated with MAbs for 30 min, 4° C. and then washed three times with cold PBSBH. Stained cells were fixed overnight in 1% paraformaldehyde (4° C.) and analyzed using a flow-cytometer, (EPICS XL-MCL, Beckman-Coulter, Miami, Fla.). Flow cytometry analysis showed that cells were strongly positive (>90%) for c-kit, moderately positive for CD13 (>60%), negative for CD14 and CD16, and slightly positive (>50%) for CD61.

D. IgE-Mediated Histamine Release From Human Mast Cells:

Histamine release assay was carried out using standard reagents and protocols obtained from Beckman Coulter (Fullerton, Calif.). Briefly, mast cells (1.5×10⁵) were sensitized with a recombinant IgE (0.1, 1 or 10 μg/ml) for 1 hr at 37° C., in the presence or absence of varying concentrations of Fcε-Fcγ proteins. The recombinant IgE consists of variable regions specific to a peptidic segment (15 mer) derived from HIV-1 gp120 V3 region and the constant region of human IgE. After washing, cells were then challenged with ovalbumin conjugated with multiple HIV-1 V3 peptides, Ova/V3 (100 ng/ml) for 2 hr at 37° C. Cell supernatants were harvested and their histamine contents measured using a histamine immunoassay kit (Beckman-Coulter, Palatine, Ill.) according to the manufacturer's protocol. The immunoassay was based on a competition between the histamine to be assayed and histamine-alkaline phosphatase conjugate. Briefly, the histamine present in the cell supernate was acylated with an acylating reagent at a slightly alkaline pH, and added onto microtiter wells coated with antibodies. Microtiter wells were coated with a limited number of antibodies allowing for a competition to take place between the conjugate and the acylated histamine in the sample. After 2 hour of incubation at 4° C., the wells were rinsed to remove unbound components. Bound enzymatic activity was measured by adding a chromogenic substrate (pNPP). The color intensity was inversely proportional to the concentration of histamine in the sample. Histamine released was calculated on the basis of a standard curve obtained with standards provided in the kit. Control groups that were included in the studies were: untreated cells, cells treated with ionophore, A23187 (2 μM), cells treated with IgE only, or cells treated with Ova/V3 only.

The foregoing description, terms, expressions, and examples are exemplary only and not limiting. The invention includes all equivalents of the foregoing embodiments, both known and unknown. The invention is limited only by the claims that follow and not by any statement in any other portion of this document or in any other source.

1. A fusion protein comprising an IgE Fcεfragment and an IgG Fcγfragment, wherein said fusion protein binds to an FcεRI and/or FcεRII receptor and an FcγRIIB receptor.

2. The fusion protein of claim 1, wherein the Fcεfragment comprises Hinge-Fcε2-Fcε3-Fcε4 or a functional fragment thereof capable of binding to FcεRI and FcεRII with at least 75% binding affinity as native IgE.

3. The fusion protein of claim 1 selected from Hinge-Fcε2-Fcε3-Fcε4-Fcγ, Fcε2-Fcε3-Fcε4-Fcγ, Fcε2-Fcε3-Fcγ, Fcε3-Fcγ, and Fcε3-Fcε4-Fcγ.

4. The fusion protein of claim 1, wherein the Fcγfragment is a fragment from an IgG subclass selected from IgG1 or IgG3, or a modified form thereof which can bind to FcγRIIB.

5. The fusion protein of claim 1, wherein the Fcγfragment is Hinge-Fcγ2-Fcγ3, Fcγ2-Fcγ3, or Fcγ2.

6. A fusion protein comprising an Fcεfragment and an Fcγfragment, wherein said fusion protein comprises Fcε2-Fcε3-Fcγ3 and binds to FcεRI and FcεRII receptors with at least 75% binding affinity as native IgE.

7. The fusion protein of claim 1, wherein the Fcεfragment and the Fcγfragment are conjugated via a linker.

8. The fusion protein of claim 7, wherein the linker is nonimmunogenic.

9. The fusion protein of claim 8, wherein the nonimmunogenic linker is a 16 amino acid linker having the sequence GGSGGSGGGGSGGGGS (SEQ ID NO.: 2).

10. A composition comprising the fusion protein of claim 1 and a physiologically acceptable excipient, diluent, or carrier.

11. A nucleic acid molecule encoding the fusion protein of claim 1.

12. The nucleic acid molecule of claim 11, wherein said nucleic acid molecule is operatively linked to a transcription control sequence.

13. A host cell transfected with the nucleic acid molecule of claim 12.

14. A method of making the fusion protein of claim 1, comprising making a vector coding for the fusion protein, transfecting a host system with the vector and expressing the fusion protein in the host system.

15. A method of making the fusion protein of claim 1, comprising conjugating the Fcε fragment to the Fcγ fragment.

16. A method of blocking the binding of IgE to FcεRI and/or FcεRII in a mammalian subject comprising administering to the mammalian subject a blocking amount of the fusion protein of claim 1.

17. The method of claim 16, wherein the FcεRI or FcεRII is crosslinked to FcγRIIB.

18. A method of inhibiting expression of FcεRI and down-regulating production of IgE in a mammalian subject, comprising administering to a mammalian subject an inhibiting amount of the fusion protein of claim 1.

19. A method of ameliorating or preventing an IgE-mediated allergic response in a susceptible mammalian subject, comprising administering to the mammalian subject an ameliorating or preventing amount of the fusion protein of claim 1.

20. The method of claim 19, wherein the allergic response is associated with allergic asthma, allergic rhinitis, hay fever, food allergy, atopic dermatitis and drug allergy.

21. The method of claim 20, wherein the allergic response is caused by peanut allergen.

22. The method of claim 19, further comprising the administration of an anti-IgE antibody or fragment thereof, wherein said administration is simultaneous, separate, or sequential.

23. The method of claim 22, wherein the allergic response is associated with asthma, allergic rhinitis, hay fever, food allergy, atopic dermatitis and drug allergy.

24. A method of ameliorating or preventing an IgE-mediated allergic disease in a mammalian subject, comprising administering an ameliorating or preventing amount of the fusion protein of claim 1.

25. The method of claim 24, wherein the fusion protein or composition and allergen are administered separately, sequentially, or simultaneously.

26. The method of claim 19, wherein the mammalian subject is a human, a canine, or a feline.
